(19) United States
(12) Patent Application Publication
(10) Pub. No.: US 2010/0191025 A1
(43) Pub. Date: Jul. 29, 2010

(54) PROCESS FOR PREPARING (HYDRO)(CHLORO)OLEFINS

(75) Inventor: Sylvain Perdrieux, Charly (FR)

Correspondence Address: ARKEMA INC.;PATENT DEPARTMENT - 26TH FLOOR, 2000 MARKET STREET, PHILADELPHIA, PA, 19103-3222, US

(73) Assignee: Arkema France, Colombes (FR)

(21) Appl. No.: 12/601,365

(22) PCT Filed: May 13, 2008
(86) PCT No.: PCT/FR2008/050821

§ 371 Date: Nov. 23, 2009

(30) Foreign Application Priority Data
May 31, 2007 (FR) 0755357

(51) Int. Cl. C07C 17/25; C07C 17/087

(52) U.S. Cl. 570/155; 570/168; 570/166; 570/165

(57) ABSTRACT

The present invention relates to a process for preparing (hydro)(chloro)fluoroolefins comprising at least one step of fluorination in the liquid phase of a (hydro)haloalkane or of a (hydro)haloalkene in the presence of at least one ionic liquid as a catalyst. The ionic liquids are derivatives of Lewis acids based on aluminum, titanium, niobium, tantalum, tin, antimony, nickel, zinc or iron.

The present invention relates to a process for preparing (hydro)(chloro)fluoroolefins. One subject of the present invention is more particularly a process for preparing (hydro)(chloro)fluoropropenes.

Document JP 4110388 describes the use of hydrofluoropropenes of formula C₃HₘFₙ, with m, n representing an integer between 1 and 5 inclusive and m+n=6, as a heat transfer fluid, in particular tetrafluoropropene and trifluoropropene.

Quite recently, document WO 2004/037913 teaches the use of tetrafluoropropene and pentafluoropropene as refrigerants having a low GWP (low global warming potential).

Hydrofluoroolefins are in general obtained by the dehydrohalogenation reaction. Thus, pentafluoropropene (CF₃CH═CF₂) is obtained either from monochloropentafluoropropane by removing one molecule of HCl or from hexafluoropropane by removing one molecule of HF (WO 05/030685, WO 98/33755).

Tetrafluoropropene may also be obtained by the dehydrofluorination reaction of pentafluoroethane (U.S. Pat. No. 5,986,151).

Trifluoropropene and tetrafluoropropene are also formed during the fluorination reaction of pentachloroethane with HF in the presence of a catalyst (WO 98/12161).

Another route for preparing trifluoropropene and/or tetrafluoropropene consists in reacting trichloropropene with HF in the presence of a catalyst (U.S. Pat. No. 5,811,603).

Furthermore, liquid-phase fluorination reactions necessitate, in order to be effective, using a reaction medium that is rich in HF and SbCl₅ (or SbClₓF_{y}) and temperatures between 80 and 120° C. Anhydrous HF in the form of a liquid phase forms, with SbCl₅, a very corrosive superacidic medium.

The present invention describes a process for preparing (hydro)(chloro)fluoroolefins,

of general formula (I) CₐF_{b}Cl_{c}H_{2a-b-c} with a representing an integer between 3 and 6, b representing an integer between 1 and 2a, c representing either the value zero or an integer between 1 and (2a−1),

comprising at least one step of liquid-phase fluorination of a (hydro)haloalkane of general formula (II) CₐF_{b′}X_{c}H_{2a+2-b′-c′} with a having the same meaning as in formula (I), b′ representing either the value zero or an integer between 1 and 2a−1 with b>b′, X representing an atom of Cl, Br or I and c′ representing an integer between 1 and 2a+2; when X represents an atom of Cl, c′>c;

or a (hydro)haloalkene of general formula (III) CₐF_{b″}X_{c′}H_{2a-b″-c″} with a having the same meaning as in formula (I), b″ representing either the value zero or an integer between 1 and 2a−1 with b>b″, X representing an atom of Cl, Br or I and c″ representing an integer between 1 and 2a; when X represents an atom of Cl, c″>c,

in the presence of at least one ionic liquid as a catalyst.

The (hydro)haloalkane of general formula (II) may originate from a telomerization reaction between a haloalkane preferably having one carbon atom and a haloalkene,

Preferably, a represents an integer equal to 3 or 4 and advantageously a is equal to 3.

The preferred (hydro)(chloro)fluoroolefins are trifluoropropene (CF₃CH═CH₂), chlorotrifluoropropene (CF₃CH═CHCl), 1,1,1,3-tetrafluoropropene (CF₃CH═CHF) and its isomers, 1,1,1,2-tetrafluoropropene (CF₃CF═CH₂), 1,1,1,3,3-pentafluoropropene (CF₃CH═CF₂) and 1,1,1,2,3-pentafluoropropene (CF₃CF═CHF).

The fluorination step is advantageously carried out in the presence of anhydrous hydrofluoric acid.

The process according to the invention may also comprise a step of dehydrohalogenation or of hydrofluorination of the fluorinated product or products resulting from the reaction in the presence of at least one ionic liquid.

According to one preferred embodiment of the invention, a (hydro)haloalkane of general formula (II), with a equal to 3, b′ equal to zero, c′ equal to 5 or 6 and X preferably representing Cl, is reacted with anhydrous hydrofluoric acid in the presence of a catalyst comprising at least one ionic liquid in order to give a fluorinated compound of formula C₃HₙF₈₋ₙ with n equal to 2 or 3. The fluorinated compound, after optional separation, is then subjected to a dehydrofluorination step in order to give the desired hydrofluoroolefin.

According to another preferred embodiment of the invention, a (hydro)haloalkene of general formula (III) with a equal to 3, b″ equal to zero, c″ equal to 4 and X preferably representing Cl, is reacted with anhydrous hydrofluoric acid in the presence of a catalyst comprising at least one ionic liquid in order to give a fluorinated compound of formula C₃H₂FₚX₄₋ₚ, p representing a value equal to 3 or 4, and/or of formula C₃HₙF_{8-b} with n possibly taking the value 2 or 3. When the products resulting from the fluorination step comprise a compound of formula C₃HₙF₈₋ₙ the latter is subjected to a dehydrofluorination step.

Thus, 2-chloro-1,1,1-trifluoropropene (1233 xf) and/or 1,1,1,2-tetrafluoropropene (1234 yf) may be obtained from 1,1,2,3-tetrachloropropene (1230 xa) by liquid-phase fluorination in the presence of at least one ionic liquid as a catalyst.

1,1,1,2-Tetrafluoropropene (1234 yf) may also be obtained from 2-chloro-1,1,1-trifluoropropene (1233 xf) by liquid-phase fluorination in the presence of at least one ionic liquid as a catalyst in order to give 2-chloro,1,1,1,2-tetrafluoropropane (244 bb) and/or 1,1,1,2,2-pentafluoropropane (245 bb) which is or are then subjected to a step of dehydrohalogenation either in the liquid phase or in the gas phase.

Similarly, 1,1,1,3-tetrafluoropropene (1234 ze) may be obtained from 3-chloro-1,1,1-trifluoropropene (1233 zd).

Furthermore, 1,1,1,2-tetrafluoropropene (1234 yf) may be obtained from 1,1,1,2-tetrachloro,2-fluoropropane (241 bb) by liquid-phase fluorination in the presence of at least one ionic liquid as a catalyst.

The ionic liquids which may be suitable are derivatives of Lewis acids based on aluminum, titanium, niobium, tantalum, tin, antimony, nickel, zinc or iron.

The expression “ionic liquids” is understood to mean non-aqueous salts having an ionic character that are liquid at moderate temperatures (preferably below 120° C.). The ionic liquids preferably result from the reaction between an organic salt and an inorganic compound.

The ionic liquids are preferably obtained by reaction of at least one halogenated or oxyhalogenated Lewis acid based on aluminum, titanium, niobium, tantalum, tin, antimony, nickel, zinc or iron with a salt of general formula Y⁺A⁻, in which A⁻ denotes a halide (bromide, iodide and preferably chloride or fluoride) anion or hexafluoroantimonate (SbF₆⁻) anion and Y⁺ a quaternary ammonium cation, quaternary phosphonium cation or ternary sulfonium cation.

The halogenated Lewis acid based on aluminum, titanium, niobium, tantalum, antimony, nickel, zinc or iron may be a chlorinated, brominated, fluorinated or mixed derivative, for example a chlorofluorinated acid. Mention may more particularly be made of the chlorides, fluorides or chlorofluorides of the following formulae:

TiClₓF_{y} with x+y=4 and 0≦x≦4

TaClₓF_{y} with x+y=5 and 0≦x≦5

NbClₓF_{y} with x+y=5 and 0≦x≦5

SnClₓF_{y} with x+y=4 and 1≦x≦4

SbClₓF_{y} with x+y=5 and 0≦x≦5

AlClₓF_{y} with x+y=3 and 0≦x≦3

NiClₓF_{y} with x+y=2 and 0≦x≦2

FeClₓF_{y} with x+y=3 and 0≦x≦3

As examples of such compounds, mention may be made of the following compounds: TiCl₄, TiF₄, TaCl₅, TaF₅, NbCl₅, NbF₅, SbCl₅, SbCl₄F, SbCl₃F₂, SbCl₂F₃, SbClF₄, SbF₅ and mixtures thereof. Use is preferably made of the following compounds: TiCl₄, TaCl₅+TaF₅, NbCl₅+NbF₅, SbCl₅, SbFCl₄, SbF₂Cl₃, SbF₃Cl₂, SbF₄Cl, SbF₅ and SbCl₅+SbF₅. The antimony compounds are more particularly preferred.

As examples of oxyhalogenated Lewis acids that can be used according to the invention, mention may be made of TiOCl₂, TiOF₂ and SbOClₓF_{y} (x+y=3).

In the Y⁺A⁻salt, the Y⁺ cation may correspond to one of the following general formulae:

R¹R²R³R⁴N⁺

R¹R²R³R⁴P⁺

R¹R²R³S⁺

in which the symbols R¹ to R⁴, which are identical or different, each denote a hydrocarbyl, chlorohydrocarbyl, fluorohydrocarbyl, chlorofluorohydrocarbyl or fluorocarbyl group having from 1 to 10 carbon atoms, which is saturated or unsaturated, cyclic or non-cyclic, or aromatic, one or more of these groups possibly also containing one or more heteroatoms such as N, P, S or O.

The ammonium, phosphonium or sulfonium cation Y⁺ may also be part of a saturated or unsaturated, or aromatic heterocycle having from 1 to 3 nitrogen, phosphorus or sulfur atoms, and may correspond to one or the other of the following general formulae:

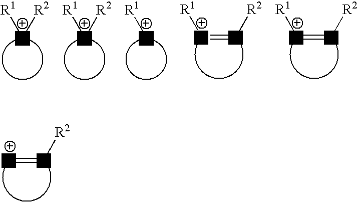

in which R¹ and R² are as defined previously.

A salt containing 2 or 3 ammonium, phosphonium or sulfonium sites in its formula may also be suitable.

As examples of Y⁺A⁻ salts, mention may be made of tetraalkylammonium chlorides and fluorides, tetraalkylphosphonium chlorides and fluorides, and trialkylsulfonium chlorides and fluorides, alkylpyridinium chlorides and fluorides, dialkylimidazolium chlorides, fluorides and bromides and trialkylimidazolium chlorides and fluorides. More particularly appreciated are trimethylsulfonium fluoride or chloride, N-ethylpyridinium chloride or fluoride, N-butylpyridinium chloride or fluoride, 1-ethyl-3-methylimidazolium chloride or fluoride and 1-butyl-3-methylimidazolium chloride or fluoride.

The ionic liquids may be prepared in a manner that is known per se by mixing, in an appropriate manner, the halogenated or oxyhalogenated Lewis acid and the organic salt Y⁺A⁻. Reference may especially be made to the method described in document WO 01/81353.

The ionic liquids that are advantageously preferred are those resulting from a Lewis acid/organic salt molar ratio that is strictly greater than 1:1.

The step of liquid-phase fluorination using, as catalyst, an ionic liquid may be carried out in batch mode, semi-continuously and continuously. When the fluorination step is carried out in batch mode, the molar amount of HF to the molar amount of starting product is between 2 and 50 and preferably between 10 and 30.

When the fluorination is carried out continuously, the molar amount of HF supplied to the molar amount of starting product supplied is at least equal to the stoichiometric ratio.

The amount of catalyst depends on the operating conditions, on the reaction medium (in the case of a continuous process) but also on the intrinsic activity of the catalyst. This amount is between 0.5 and 90 (mol) % of the reaction medium.

When the catalyst used is based on antimony, it may sometimes be advantageous to introduce chlorine in order to keep the antimony in the +5 degree of oxidation.

The temperature at which the fluorination reaction (under batch and continuous conditions) is carried out is generally between 30 and 180° C., preferably between 80 and 130° C.

The pressure at which the reaction is carried out in semi-continuous or continuous modes is chosen so as to keep the reaction medium in the liquid phase. It is generally situated between 5 and 50 bar and preferably between 10 and 40 bar; under continuous conditions, if HF constitutes the reaction medium, the operating pressure chosen is in general the saturation vapor pressure of the HF at the desired reaction temperature. The temperature of the condenser is set as a function of the amount and of the nature of the products likely to be discharged during the reaction. It is generally between −50 and 150° C. and preferably between 0 and 100° C.

A reactor made of stainless steel or of MONEL, INCONEL or HASTELLOY type alloys may be suitable for the fluorination. Compared to a conventional catalyst, the step of fluorination in the presence of an ionic liquid is less corrosive.

1. A process for preparing (hydro)(chloro)fluoroolefins, of general formula (I) CₐF_{b}Cl_{c}H_{2a-b-c} with a representing an integer between 3 and 6, b representing an integer between 1 and 2a, c representing either the value zero or an integer between 1 and (2a−1),
comprising at least one step of liquid-phase fluorination of a (hydro)haloalkane of general formula (II) CₐF_{b′}X_{c′}H_{2a+2-b′-c′}, b′ representing either the value zero or an integer between 1 and 2a−1 with b>b′, X representing an atom of Cl, Br or I and c′ representing an integer between 1 and 2a+2; when X represents an atom of Cl, c′>c;
or a (hydro)haloalkene of general formula (III) CₐF_{b″}X_{c″}H_{2a-b″-c″}, b″ representing either the value zero or an integer between 1 and 2a−1 with b>b″, X representing an atom of Cl, Br or I and c″ representing an integer between 1 and 2a; when X represents an atom of Cl, c″>c,
in the presence of at least one ionic liquid as a catalyst.

2) The process as claimed in claim 1, characterized in that a represents an integer equal to 3 or 4.

3) The process as claimed in claim 1, characterized in that the (hydro)(chloro)fluoroolefins is selected from the group consisting of trifluoropropene (CF₃CH═CH₂), chlorotrifluoropropene (CF₃CH═CHCl), 1,1,1,3-tetrafluoropropene (CF₃CH═CHF) and its isomers, 1,1,1,2-tetrafluoropropene (CF₃CF═CH₂), 1,1,1,3,3-pentafluoropropene (CF₃CH═CF₂) and 1,1,1,2,3-pentafluoropropene (CF₃CF═CHF).

4) The process as claimed in claim 1, characterized in that the process further comprises a step of dehydrohalogenation or of hydrofluorination of the fluorinated product or products resulting from the reaction in the presence of at least one ionic liquid.

5) The process as claimed in claim 4, characterized in that the ionic liquid is a derivative of Lewis acids based on aluminum, titanium, niobium, tantalum, tin, antimony, nickel, zinc or iron.

6) The process as claimed in claim 1, further characterized in that a (hydro)haloalkane of general formula (II), with a equal to 3, b′ equal to zero, c′ equal to 5 or 6, is reacted with anhydrous hydrofluoric acid in the presence of a catalyst comprising at least one ionic liquid in order to give a fluorinated compound of formula C₃HₙF₈₋ₙ with n equal to 2 or 3; and the fluorinated compound, after optional separation, is then subjected to a dehydrofluorination step.

7) The process as claimed in claim 1, further characterized in that a (hydro)haloalkene of general formula (III) with a equal to 3, b″ equal to zero, c″ equal to 4, is reacted with anhydrous hydrofluoric acid in the presence of a catalyst comprising at least one ionic liquid in order to give a fluorinated compound of formula C₃H₂FₚX₄₋ₚ, p representing a value equal to 3 or 4, and/or of formula C₃HₙF₈₋ₙ with n taking the value 2 or 3.

8) The process as claimed in claim 7, characterized in that the compound of formula C₃HₙF₈₋ₙ is subjected to a dehydrofluorination step.

9) The process as claimed in claim 6 characterized in that X represents Cl.

10) The process as claimed in claim 7 characterized in that X represents Cl.
